(19) United States
(12) Patent Application Publication
(10) Pub. No.: US 2011/0014286 A1
(43) Pub. Date: Jan. 20, 2011

(54) MIXTURE FOR PRODUCING RAPIDLY DISINTEGRATING TABLETS

(76) Inventors: Jorg Kowalczyk, Eisenberg/Steinborn (DE); Oliver Luhn, Birkenheide (DE)

Correspondence Address: OSTROLENK FABER GERB & SOFFEN, 1180 AVENUE OF THE AMERICAS, NEW YORK, NY, 100368403, US

(21) Appl. No.: 12/920,502

(22) PCT Filed: Feb. 17, 2009
(86) PCT No.: PCT/EP09/01091

§ 371 Date: Sep. 1, 2010

(30) Foreign Application Priority Data
Mar. 3, 2008 (DE) 10 2008 012 295.5

(51) Int. Cl. A61K 9/00; A61K 31/7016; A61K 9/36

(52) U.S. Cl. 424/479; 514/53; 424/400

(57) ABSTRACT

An isomalt-containing mixture including a) 60-97% by weight of agglomerated isomalt, b) 1-25% by weight of crosslinked polyvinylpyrrolidone, c) 0.1-15% by weight of lubricant, d) 1-15% by weight of water-insoluble, film-forming polymers, e) 0-15% by weight of water-soluble polymers and f) 0-15% by weight of pharmaceutical auxiliaries, where the sum of components a) to f) is 100% by weight. Also processes for the preparation of the isomalt-containing mixture and tablets comprising the mixture.

The present invention relates to an isomalt-containing mixture, preferably in the form of agglomerates, for producing rapidly disintegrating tablets, comprising agglomerated isomalt, crosslinked polyvinylpyrrolidone and water-insoluble polymers.

Tablets which disintegrate rapidly in the mouth are becoming increasingly important for the oral administration of medicaments. Such tablets have to disintegrate within a short time in the oral cavity, have a pleasant taste and must not leave behind a sandy feel. Furthermore, they should be easy to produce, with direct tableting having considerable advantages over wet granulation. Moreover, the tablets should have high mechanical strength so that they withstand packaging procedures, transportation and also pressing out from packaging without damage.

The products and processes described hitherto do not meet these requirements or do so only very inadequately.

Rapidly disintegrating tablets often consist of sugar and sugar alcohols, effervescent systems, microcrystalline cellulose and other non-water-soluble fillers, calcium hydrogenphosphate, cellulose derivatives, corn starch or polypeptides. Additionally, water-soluble polymers, customary disintegrants, such as crosslinked PVP, sodium salt and calcium salt of crosslinked carboxymethylcellulose, sodium salt of carboxymethyl starch, low-substituted hydroxypropylcellulose L-HPC, and essentially inorganic water-insoluble constituents, such as silicas, silicates, inorganic pigments, are used. Furthermore, the tablets can also comprise surfactants.

EP 0 028 905 B1 discloses tablets comprising isomaltulose and processes for producing them. The specification discloses an advantageous use of isomaltulose as diluent for producing compressed products since isomaltulose can be compressed directly without binders and without controlled granulation. According to this specification, crystallized isomaltulose prepared from the enzymatic conversion of sucrose to isomaltulose is used directly for the tableting.

DE 196 39 343 C2 discloses compressed products comprising isomalt and isomalt variants. Production of the compressed products takes place by simple compression of the individual components without providing a specific mechanical and/or chemical treatment of the individual components.

WO 01/19401 and EP 1 214 093 B1 describe a process for producing a compressed product made of isomaltulose, isomalt or isomalt variants. By virtue of this process, it is possible to produce compressed products which can lead to high tablet hardnesses even at low compressive pressures. At the same time, these compressed products are characterized by improved sensory properties.

WO 2003/051338 discloses a directly tabletable and readily compressible auxiliary formulation which comprises mannitol and sorbitol.

US 2002/0071864 A1 discloses a tablet which disintegrates in the oral cavity within 60 seconds and is formulated primarily from a physical mixture of spray-dried mannitol and a coarse-grained crosslinked polyvinylpyrrolidone and also a limited selection of active ingredients. These tablets have a breaking strength of ca. 40 N and produce an unpleasant, sandy mouth feel.

According to U.S. Pat. No. 6,696,085 B2, a type C methacrylic acid copolymer is intended to be used as disintegration agent. Besides a low breaking strength (<20 N), the tablets have a high friability (>7%) and include a high fraction in the region of 15% by weight of a coarse-grained disintegrant. Consequently, they have low mechanical strength, and on account of the high fraction of coarse-grained disintegrant, produce an unpleasant, sandy mouth feel.

WO 2007/071581 A2 discloses a pharmaceutical formulation for producing rapidly disintegrating tablets. This pharmaceutical formulation includes inter alia agglomerates made of sugar or sugar alcohols, such as trehalose, mannitol, erythritol or sorbitol. The tablets produced by this pharmaceutical formulation are characterized by very rapid disintegration times following oral administration.

A disadvantage of the processes described hitherto for producing tablets, however, is that these tablets are still characterized by disintegration times that are too slow and a mechanical strength that is too low and are in need of improvement in terms of their sensory properties.

The present invention is therefore based on the technical problem of providing tablets which firstly rapidly disintegrate following oral administration and secondly are characterized by high mechanical stability, in particular improved abrasion resistance and relatively high tablet hardness.

The present invention solves the technical problem on which it is based through the provision of a mixture comprising
a) 60 to 97% by weight of agglomerated isomalt,
b) 1 to 25% by weight of crosslinked polyvinylpyrrolidone (also referred to as PVP),
c) 0.1 to 15% by weight, preferably 1 to 15% by weight, of lubricant, in particular magnesium stearate (Mg stearate),
d) 1 to 15% by weight of water-insoluble, film-forming polymers,
e) 0 to 15% by weight of water-soluble polymers and
f) 0 to 15% by weight of further pharmaceutical auxiliaries,
   where the sum of components a) to f) is 100% by weight (based on dry substance of the total mixture).

Such an isomalt-containing mixture of the present invention is particularly suitable as a pharmaceutical formulation, and can accordingly comprise, in an advantageous and preferred embodiment, besides constituents a) to f), active ingredients, in particular pharmaceutical active ingredients.

Furthermore, according to the invention, a process for producing such a mixture is also provided. In particular, the present invention also relates to tablets which comprise, essentially comprise or consist of, a mixture according to the invention.

Surprisingly, it has been established that the tablets prepared using the mixture according to the invention which have agglomerated isomalt have, following oral administration, extremely rapid disintegration times of less than 40 seconds, preferably less than 30 seconds, particularly preferably less than 20 seconds. Furthermore, it has been established that these tablets have proven to be particularly stable. The tablets according to the invention are therefore characterized by a high abrasion resistance and also by high mechanical stability.

The mixture according to the invention comprises, as component a), 60 to 97% by weight, preferably 70 to 95% by weight, particularly preferably 75 to 93% by weight, in particular 85% to 95% by weight, of agglomerated isomalt.

In connection with the present invention, the term isomalt is understood as meaning a mixture that comprises 6-O-α-D-glucopyranosyl-D-sorbitol (1,6-GPS) and 1-O-α-D-glucopyranosyl-D-mannitol (1,1-GPM), in particular isomalt ST, isomalt GS, an isomalt variant and in particular 1-O-α-D-glucopyranosyl-D-sorbitol (1,1-GPS)-containing mixtures of 1,1-GPM and 1,6-GPS.

Isomalt ST, also known under the name Palatinit®, refers to a virtually equimolar mixture of the two stereoisomers 6-O-α-D-glucopyranosyl-D-sorbitol (1,6-GPS) and 1-O-α-D-glucopyranosyl-D-mannitol (1,1-GPM). According to the invention, it is provided in a preferred embodiment that the isomalt is isomalt ST or isomalt GS. Isomalt ST refers to a mixture of 53 to 47% by weight of 1,6-GPS and 47 to 53% by weight of 1,1-GPM. Isomalt GS refers to a mixture of 60 to 80% by weight, in particular 75% by weight, of 1,6-GPS and 20 to 40% by weight, in particular 25% by weight, of 1,1-GPM. Isomalt variants may be mixtures of 10 to 50% by weight of 1,6-GPS, 2 to 20% by weight of 1,1-GPS and 30 to 70% by weight of 1,1-GPM or mixtures of 5 to 10% by weight of 1,6-GPS, 30 to 40% by weight of 1,1-GPS and 45 to 60% by weight of 1,1-GPM. Isomalt variants may also be 1,6-GPS- or 1,1-GPM-enriched mixtures. 1,6-GPS-enriched mixtures are characterized by a 1,6-GPS fraction of from 57 to 99% by weight and a 1,1-GPM fraction of from 43 to 1% by weight, whereas 1,1-GPM-containing mixtures are characterized by a 1,6-GPS fraction of from 1 to 43% by weight and a 1,1-GPM fraction of from 57 to 99% by weight.

To produce agglomerated isomalt, the isomalt used, thus for example isomalt ST or isomalt GS, is firstly, preferably in dry form, ground or pounded. In a further process step, a fraction having a certain maximum size of the particles present therein is separated off. The separated-off ground fraction is then mixed with a liquid binder, for example water, an isomalt solution or isomalt suspension or Kollidon. Furthermore, preference is also given to binders in alkaline or aqueous solution, as are customary for example in the production of wet granules in the pharmaceutical sector. Particular preference is given to cellulose derivatives with methylation, ethylation, hydroxypropylation, sulfonation, nitration or acetylation as possible modifications of cellulose, such as HPMC, MC, HPC, NaCMC, EC, modified starch, for example from corn, wheat or potato.

The liquid binder can be introduced into the ground fraction for example by means of spraying. It is of course also possible to introduce the ground isomalt into the liquid binder through spraying or to bring the two into contact with one another simultaneously by spraying. The formation of the agglomerates preferably takes place by means of a fluidized-bed agglomerator.

In a preferred embodiment, the agglomerated isomalt is in finely divided form with maximum particle sizes of 100 μm, preferably at most 50 μm, in particular with a maximum size of 40 μm, preferably at most 35 μm and particularly preferably at most 30 μm. If appropriate, it may be provided to adjust the particle size through grinding.

In connection with the present invention, a particle diameter is also understood as meaning the particle size, where a particle size or a particle diameter of for example at most 100 μm means that at least 90% of the particles have a diameter of at most 100 μm.

In a particularly preferred embodiment, the isomalt-containing mixture of the present invention is present for its part in the form of an agglomerate, comprising the components a) to d) and optionally e) and f). In a particularly preferred embodiment, the agglomerates of this isomalt-containing mixture have a size of from 0.063 to 500 μm.

In a particularly preferred embodiment, it is provided that the isomalt-containing mixture, in particular tablets comprising the isomalt-containing mixture, comprise isomalt as the sole sweetener, i.e. no further sweeteners such as sugar, sugar alcohols, sugar substitutes or intense sweeteners are present in the mixture or the tablet. In a further preferred embodiment, it is provided that the isomalt-containing mixture, in particular the tablets comprising this mixture, comprise isomalt as the sole sugar alcohol. In a further preferred embodiment, it is provided that the isomalt-containing mixture, in particular the tablets comprising this mixture, comprise isomalt as the sole bodying sweetener. In a further preferred embodiment, it is provided that the isomalt-containing mixture, in particular the tablets comprising this mixture, is sugar-free. In a further preferred embodiment, it is provided that the isomalt-containing mixture, in particular the tablets comprising this mixture, are free from glucose, fructose, lactose and/or sucrose. In a preferred embodiment, the isomalt-containing mixtures, in particular the tablets comprising these mixtures, are suitable for diabetics, have a reduced calorific value, are friendly to teeth and/or are acariogenic.

As component b), crosslinked polyvinylpyrrolidones are used in amounts of from 1 to 25% by weight, preferably 2 to 15% by weight, particularly preferably 3 to 10% by weight. Such crosslinked polyvinylpyrrolidones are water-insoluble, but not film-forming. The crosslinked polyvinylpyrrolidone can have an average particle size of from 2 to 60 μm, preferably less than 50 μm, particularly preferably less than 30 μm. Very particular preference is given to crosslinked polyvinylpyrrolidones with a hydration capacity of greater than 6.5 g/g. Here, the hydration capacity is determined according to the following method:

2 g of polymer are weighed into a centrifuge tube and allowed to swell with 40 ml of water for 15 minutes. Then, the mixture is centrifuged for 15 minutes at 2000 rpm and the supernatant liquid is poured off as completely as possible.

$$Hydration capacity = \frac{Final weight - Tare}{Initial weight}$$

The high hydration capacity of the crosslinked polyvinylpyrrolidone leads in the mixture and the pharmaceutical formulations or tablets produced therefrom to very rapid disintegration and produces a particularly soft mouth feel.

As component c), lubricants can be used in an amount of from 0.1 to 15% by weight, preferably 1 to 15% by weight, in particular 0.1 to 5% by weight. In connection with the present invention, a lubricant is to be understood as meaning an agent which improves the compression on the tableting press and leads to reduced friction between tablet and die wall. Lubricants preferred according to the invention are also calcium stearate or aluminum stearate, stearic acid, palmitic acid, myristic acid, lauric acid or capric acid, talc, polyethylene glycol (PEG), silicates, lauryl sulfates, starches, hydrogenated vegetable oils such as Cutina or Sterotex, behenates or combinations thereof. Preferably, it is envisaged to use magnesium stearate as lubricant. It is particularly preferably envisaged to use a mixture of magnesium stearate and polyethylene glycol as lubricant, in particular to use a mixture of 0.1 to 2% by weight of magnesium stearate and 1 to 6% by weight of PEG, in particular of 0.5% magnesium stearate and 2 to 5% PEG, in particular PEG 6000, preferably 6000 P (fine, Clariant). It could be shown that such a combination of magnesium stearate and PEG, in particular PEG 6000, preferably PEG 6000 P, accelerates tablet disintegration compared with mixtures comprising merely magnesium stearate.

The preparation of the mixture preferred according to the invention in the form of agglomerates can take place by agglomeration in mixers, fluidized-bed apparatuses or spray towers. For this, solid starting materials and granulation liquid are first mixed with one another and the wet mixing material is then dried. According to the present invention, the granulating liquid used is at least an aqueous dispersion of component d), the water-insoluble polymer.

As component d), water-insoluble polymers are used in amounts of from 1 to 15% by weight, preferably 1 to 10% by weight. Preference is given to polymers which are insoluble in the pH range from 1 to 14, i.e. have a pH-independent insolubility in water at any pH. Furthermore, preference is given to polymers which are water-insoluble at any pH in the pH range from 6 to 14.

In one preferred embodiment, the polymers are film-forming polymers. In this connection, film-forming means that the polymers have in aqueous dispersion a minimum film-forming temperature of from −20 to +150° C., preferably 0 to 100° C.

Suitable and preferred polymers are polyvinyl acetate, ethylcellulose, methacrylic acid-ethyl acrylate copolymer, methyl methacrylate-ethyl acrylate copolymers, ethyl acrylate-methyl methacrylate-trimethylammonium ethyl methacrylate terpolymers, butyl methacrylate-methyl methacrylate-dimethylaminoethyl methacrylate terpolymers. The acrylate-methacrylate copolymers are described in more detail in the European Pharmacopoeia as Polyacrylate Dispersion 30%, in the USP as Ammonio-Methacrylate Copolymer and in JPE as Aminoalkyl-Methacrylate Copolymer E.

As preferred component d), polyvinyl acetate is used. This can be used as aqueous dispersion with solids contents of from 10 to 45% by weight. Moreover, preference is given to polyvinyl acetate with a molecular weight between 100 000 and 1 000 000 daltons, particularly preferably between 200 000 and 800 000 daltons.

Furthermore, the mixture according to the invention comprises water-soluble polymers in amounts of from 0 to 15% by weight as components e). Suitable water-soluble polymers are, for example, polyvinylpyrrolidones, vinylpyrrolidone-vinyl acetate copolymers, polyvinyl alcohols, polyvinyl alcohol-polyethylene glycol block copolymers, polyvinyl alcohol-polyethylene glycol graft copolymers, polyethylene glycols, ethylene glycol-propylene glycol block copolymers, hydroxypropylmethylcellulose, hydroxypropylcellulose, hydroxyethylcellulose, carrageenans, pectins, xanthans, alginates.

If appropriate, by adding pharmaceutical auxiliaries as component f) in amounts of from 0 to 15% by weight, for example acidifying agents, buffer substances, intense sweeteners, aromas, flavor enhancers and dyes, it is preferably possible to further improve taste and appearance of the tablets comprising the isomalt-containing mixture according to the invention. The following substances are especially preferred in this connection: citric acid, tartaric acid, ascorbic acid, sodium dihydrogenphosphate, cyclamate, saccharin-Na, aspartame, menthol, peppermint aroma, fruit aromas, vanilla aroma, glutamate, riboflavin, beta carotene, water-soluble dyes, finely divided color lakes. By adding thickeners such as high molecular weight polysaccharides, the mouth feel can be additionally improved by increasing the softness and the volume feel.

Preferably, surfactants may also be added as component f). Preferred surfactants are, for example, sodium lauryl sulfate, dioctyl sulfosuccinate, alkoxylated sorbitan esters such as polysorbate 80, polyalkoxylated derivatives of castor oil or hydrogenated castor oil, for example Cremophor® RH 40, alkoxylated fatty acids, alkoxylated hydroxy fatty acids, alkoxylated fatty alcohols, alkali metal salts of fatty acids and lecithins.

Moreover, to further improve the disintegration, it is also possible to add finely divided pigments because they increase the internal interfaces and consequently water can penetrate into the tablet more rapidly. These pigments, such as iron oxides, titanium dioxide, colloidal or precipitated silica, calcium carbonates, calcium phosphates, naturally have to be very finely divided, otherwise a grainy taste would again arise.

During agglomeration in the fluidized bed for producing the isomalt-containing mixture preferred according to the invention in agglomerate form, an aqueous dispersion of the water-insoluble polymer is sprayed onto a fluidizing mixture of agglomerated isomalt and crosslinked PVP, resulting in agglomeration of the fine particles. The inlet air temperatures are 30 to 100° C., and the outlet air temperatures are 20 to 70° C.

In the case of production in spray towers, the so-called FSD or SBD technology (FSD: fluidized spray drying; SBD: spray bed drying) is preferably used. Here, a suspension of the agglomerated isomalt in water is firstly spray-dried; in the lower section of the spray dryer or in an attached fluidized bed, the addition of crosslinked PVP and the spraying-in of an aqueous dispersion of the water-insoluble polymer takes place, resulting in agglomeration of the particles. Fine, if appropriate recycled, particles can furthermore be blown again in front of the spray nozzle of the agglomerated isomalt solution and thus be additionally agglomerated by spray agglomeration.

For the agglomeration process preferably provided, it is preferred to carry out a multistage spraying process. At the start, the spraying rate is preferably kept low in order to prevent overwetting of the product initial charge and consequently agglutination thereof. As the process time increases, the spraying rate can preferably be increased and thus the agglomeration tendency can be raised. It is preferred to adapt the inlet air amount and/or temperature in an appropriate manner during the process. Particularly during the drying phase, it is advantageous to reduce the amount of inlet air and thus to prevent abrasion of the agglomerates due to high mechanical stress.

The fineness of the spray droplet of the binder solution or dispersion (adjustable via the atomization gas pressure), the nozzle geometry and distance of the nozzle from the product bed may be considered further adaptation parameters for the agglomerate size of the isomalt-containing mixture in agglomerate form preferred according to the invention. The finer and more uniform the spraying, the finer and more uniform are the resulting agglomerates. The further the nozzle from the product bed, the poorer the agglomeration behavior.

Furthermore, the agglomerates preferred according to the invention can also be produced in a mixer by continuous aggregation with mixing. Such a continuous form of aggregation with mixing is the so-called “Schugi granulation”. Here, solid starting materials and the granulating liquid comprising the water-insoluble polymer are mixed with one another in a continuously operating vertically arranged high-speed mixer (see also M. Bohnet, “Mechanische Verfahrenstechnik”, Wiley VCH Verlag, Weinheim 2004, p. 198 ff.).

According to one particular embodiment, the cross-linked PVP is suspended in the aqueous dispersion of the water-insoluble polymer.

In a particularly preferred embodiment, the present invention also relates to a process for the preparation of a mixture of the present invention, in particular of a mixture which is present in agglomerate form, where agglomerated isomalt and crosslinked polyvinylpyrrolidone are agglomerated with an aqueous dispersion of the water-insoluble polymer in the presence of the lubricant. In a further preferred embodiment, the present invention relates to a process for the preparation of a mixture of the present invention, in particular of a mixture which is present in agglomerate form, where agglomerated isomalt is agglomerated with an aqueous dispersion of the water-insoluble polymer, which additionally comprises crosslinked polyvinylpyrrolidone in suspended form, in the presence of the lubricant. Advantageously, an agglomeration provided in this way can take place in a preferred embodiment in a fluidized-bed agglomerator, a mixer or a spray tower.

In the preferred mixture in agglomerate form obtained in this way, the agglomerated isomalt has a maximum particle size of 100 μm, preferably less than 50 μm, preferably less than 40 μm, preferably less than 35 μm, and particularly preferably of 30 μm. The water-insoluble, film-forming polymer serves here as agglomerating agent for agglomerating the fine isomalt particles and the particles of crosslinked PVP.

The isomalt-containing mixture present as agglomerate in a particularly preferred embodiment, comprising agglomerated isomalt, crosslinked polyvinylpyrrolidone, lubricant and water-insoluble film-forming polymers and also optionally water-soluble polymers and pharmaceutical auxiliaries, has in a preferred embodiment a particle size of from 0.063 to 500 μm, preferably from 63 to 300 μm.

The isomalt-containing mixtures according to the invention can advantageously also be used for producing tablets which are left to disintegrate in a glass of water prior to use. The production of tablets which are swallowed intact is naturally also possible.

For the production of tablets, the customary processes can be used, with direct tableting and roll compaction offering particular advantages. On account of the particular properties of the isomalt-containing mixtures according to the invention, as a rule only at least one active ingredient and the isomalt-containing mixture according to the invention are required. The tablet recipe is therefore very simple and very reproducible, and the process is easy to validate.

According to the invention, it has been found that a water-insoluble film-forming polymer together with PVP and isomalt accelerates the disintegration of tablets to a surprisingly great extent.

Furthermore, the mixtures according to the invention have extraordinarily good flowabilities and compressibilities, which lead to mechanically very stable tablets. The breaking strength of the tablets produced with the aid of the isomalt-containing mixtures according to the invention is >50 N. The breaking strengths are often above 80 N, even when using active ingredients that are difficult to compress. The friabilities are <0.2%. Consequently, damage does not arise during customary tablet handling.

On account of the agglomerated isomalt and the fine crosslinked polyvinylpyrrolidone, the tablets exhibit virtually no changes of the tablet surface when stored under humid conditions. In contrast to coarse crosslinked polyvinylpyrrolidone, there is no pimple formation due to severely swollen particles. The tablets comprising isomalt-containing mixtures according to the invention are very stable upon storage, exhibit only slight abrasion or no abrasion at all and retain their attractive appearance.

In a particularly preferred embodiment, the present invention relates to tablets comprising a mixture of the present invention. In a particularly preferred embodiment, the tablets according to the invention are characterized in that they have disintegration data in an aqueous medium of less than or equal to 25 seconds. In a further preferred embodiment, the tablets according to the invention are characterized by a breaking strength of greater than or equal to 50 N. In a further preferred embodiment, the tablets are characterized in that they have 20 to 99% by weight of a mixture of the present invention. Advantageously and in a preferred embodiment, the tablets optionally have further auxiliaries.

In a particularly preferred embodiment of the present invention, it is provided that the tablets according to the invention have a coating. In a particularly preferred embodiment, such a coating is a sugar-free coating. The invention provides advantageously and in a particularly preferred embodiment that the tablets has with a coating which comprises isomalt GS or essentially consists of this or consists of this. In a further preferred embodiment, the coating can be composed of two or more individual layers in the form of a multilayer coating. It may also be provided that the coating is an isomalt ST coating. In a preferred embodiment, it may also be provided that the coating is composed of different isomalt types, for example comprises one isomalt GS coating and one isomalt ST coating or two or more of each.

Further preferred embodiments arise from the dependent claims. The invention is explained in more detail below by reference to the examples.

### EXAMPLE 1

### Preparation of Compressed Products

Isomalt ST (standard, virtually equimolar mixture of 1,1-GPM and 1,6-GPS) was ground dry to a d₉₀=50 μm in a classifier mill. The procedure was likewise carried out with isomalt GS (composition ca. 76% 1,6-GPS and 24% 1,1-GPM).

To produce the agglomerates, a fluidized-bed agglomerator was used in the batch process, specifically the STREA 7 agglomerator from Aeromatic. The experimental batches were in each case 10 kg. Here, the ground bulk material was placed in the fluidized-bed agglomerator and a fluidized bed was built up at ca. 60° C. Upon reaching this temperature, a ca. 75° C. hot binder solution was sprayed into the fluidized bed, 3% by weight of Kollidon 30 being used. The spraying pressure used as between 2.0 and 4.5 bar, a prepressure of from 0.4 to 0.8 bar being used. The agglomerates formed were then dried at a constant inlet air temperature of ca. 80° C. up to an outlet air temperature of ca. 60° C., product cooling taking place with outside air. Then, by means of a tumbler screening machine with a screen lining of 0.8 mm to 0.1 mm, a size fractionation was carried out. Oversize particles and dusts were separated off in the process.

### EXAMPLES 2 TO 7

Examples 2 to 7 show the disintegration-promoting effect of polyvinyl acetate as water-insoluble polymer compared with water-soluble polymers.

Firstly, agglomerates were prepared from agglomerated isomalt according to example 1 and according to table 1 below in the fluidized bed: agglomerated isomalt and crosslinked PVP were introduced as initial charge and agglomerated with aqueous binder solutions/dispersions, which were sprayed into the fluidized-bed granulator (Glatt, GPCG3.1) by means of topspray methods.

The preparation was carried out by a two-stage agglomeration process, with a relatively low spraying rate firstly being chosen and the spraying rate then being increased.

The following preparation conditions were used in a two-stage agglomeration process:

Batch size: 1 kg
Concentration of the binder solution/-dispersion: 10% by weight
Inlet air temperature: 55° C.
Outlet air temperature at the start: 35° C.
Outlet air temperature after adjusting the spraying rate: 25° C.
Spraying rate at the start: 7.5 g/min
Spraying rate following adjustment: 20 g/min

TABLE 1
Formulation composition of agglomerates 2 to 7 in % by weight

| | 2 | 3 | 4 | 5 | 6 | 7 |
|---|---|---|---|---|---|---|
| Isomalt ST, agglomerated | 93 | 93 | — | — | — | — |
| Isomalt GS, agglomerated | — | — | 90 | 90 | 90 | 90 |
| Crosslinked PVP | 3.0 | 3.0 | 4.5 | 4.5 | 4.5 | 4.5 |
| (Kollidon CL) | | | | | | |
| PVP (Kollidon 30) | 3.0 | — | 4.5 | — | — | — |
| Polyvinyl alcohol/ | — | — | — | 4.5 | — | — |
| polyethylene | | | | | | |
| glycol block copolymer | | | | | | |
| (Kollicoat IR) | | | | | | |
| Methacrylic acid/ethyl | — | — | — | — | 4.5 | — |
| acrylate copolymer | | | | | | |
| (Kollicoat MAE 100 P) | | | | | | |
| Polyvinyl acetate | — | 3.0 | — | — | — | 4.5 |
| Magnesium stearate | 1.0 | 1.0 | 1.0 | 1.0 | 1.0 | 1.0 |

The agglomerates prepared in this way were mixed with the lubricant magnesium stearate in a Turbula mixer for 5 min. These mixtures were then tableted on a fully instrumented rotary tablet press (Korsch PH 100/6) at a rotational speed of 30 rpm. The rotary tablet press was equipped with 6 punches (10 mm, biplanar, faceted). The tablet weight was set to 300 mg. First, tableting was carried out at a compressive force of 18 kN. The tablets differed in hardness depending on the compressibility of the powder. The compression force was then adjusted in each case such that the breaking strength of the tablets was 60 N.

The tablets were investigated with regard to breaking strength (tablet tester HT-TMB-CI-12 F, Kraemer), friability (Roche-Friabilator, Erweka) and disintegration time in phosphate buffer pH 7.2 (disintegration tester ZT 74, Erweka). The numerical data to the left of the oblique stroke relate to the tablets which were obtained using a compressive force of 18 kN.

TABLE 2
Tablet properties for formulations 2 to 7

| | Breaking strength | Friability | Disintegration time |
|---|---|---|---|
| | [N] | [%] | [s] |
| 2 | 180/60 | 0.35/0.40 | 120/75 |
| 3 | 180/60 | 0.30/0.40 | 45/20* |
| 4 | 200/60 | 0.40/0.45 | 180/120 |
| 5 | 250/60 | 0.40/0.50 | 210/150 |
| 6 | 220/60 | 0.30/0.50 | 240/180 |
| 7 | 200/60 | 0.30/0.40 | 60/20* |

*Determination of disintegration times <20 s is not possible for reasons of endpoint detection.

### EXAMPLES 8 TO 11

Examples 8 to 11 show the suitability of a rapidly disintegrating auxiliary in an active ingredient formulation.

The rapidly disintegrating auxiliary is prepared by agglomeration in the fluidized bed of agglomerated isomalt ST (90% by weight) and crosslinked PVP (4.5% by weight) with polyvinyl acetate (4.5% by weight). The direct tableting agent prepared in this way was mixed with 1.0% by weight of lubricant (Mg stearate), the amount of active ingredient stated in Table 3 was metered in and then compressed on a rotary tablet press (Korsch PH 100/6) to give tablets with a breaking strength of 60 N.

TABLE 3
Active ingredient, amount of active ingredient, tablet weight
and diameter of active ingredient formulations 8 to 11

| | Active | Amount of | Tablet | |
|---|---|---|---|---|
| | ingredient | active ingredient | weight | Diameter |
| 8 | Loratadine | 10 mg | 250 mg | 8 mm |
| 9 | Loperamide HCl | 2 mg | 100 mg | 6 mm |
| 10 | Cetirizine 2HCl | 10 mg | 280 mg | 10 mm |
| 11 | Lorazepam | 2 mg | 120 mg | 7 mm |

The tablets were investigated with regard to breaking strength (tablet tester HAT-TMB-CI-12 F, Kraemer), friability (Roche Friabilator, Erweka) and disintegration time in phosphate buffer pH 7.2 (disintegration tester ZT 74, Erweka).

TABLE 4
Tablet properties for formulations 8 to 11

| | Breaking strength | Friability | Disintegration time |
|---|---|---|---|
| | [N] | [%] | [s] |
| 8 | 60 | 0.45 | 30 |
| 9 | 60 | 0.45 | 20* |
| 10 | 60 | 0.45 | 25 |
| 11 | 60 | 0.45 | 20* |

*Determination of disintegration times <20 s is not possible for reasons of endpoint detection.

1. An isomalt-containing mixture, comprising:
a) 60-97% by weight of agglomerated isomalt,
b) 1-25% by weight of crosslinked polyvinylpyrrolidone,
c) 0.1-15% by weight of lubricant,
d) 1-15% by weight of water-insoluble, film-forming polymers,
e) 0-15% by weight of water-soluble polymers, and
f) 0-15% by weight of at least one pharmaceutical auxiliary,
wherein the sum of the components a) to f) is 100% by weight.

2. The mixture according to claim 1, wherein the agglomerated isomalt is agglomerated isomalt ST or agglomerated isomalt GS.

3. The mixture according to claim 2, wherein isomalt ST comprises 6-O-α-D-glucopyranosyl-D-sorbitol (1,6-GPS) in an amount of 47-53% by weight and 1-O-α-D-glucopyranosyl-D-mannitol (1,1-GPM) in an amount of 53-47% by weight (in each case based on dry substance of the isomalt).

4. The mixture according to claim 2, wherein isomalt GS comprises 6-O-α-D-glucopyranosyl-D-sorbitol (1,6-GPS) in an amount of 60-80% by weight, and 1-O-α-D-glucopyranosyl-D-mannitol (1,1-GPM) in an amount of 40-20% by weight (in each case based on dry substance of the isomalt GS).

5. The mixture according to claim 1, wherein a maximum particle size of the isomalt is 100 μm.

6. The mixture according to claim 1, comprising a crosslinked polyvinylpyrrolidone with an average particle size of less than or equal to 50 μm.

7. The mixture according to claim 1, comprising a crosslinked polyvinylpyrrolidone with a hydration capacity of greater than 6.5 g/g.

8. The mixture according to claim 1, wherein the lubricant comprises magnesium stearate.

9. The mixture according to claim 1, wherein the water-insoluble film-forming polymer is polyvinyl acetate.

10. The mixture according to claim 1, wherein the water-insoluble film-forming polymer is polyvinyl acetate in the form of an aqueous dispersion.

11. The mixture according to claim 1, wherein the water-soluble polymer is polyvinylpyrrolidone.

12. The mixture according to claim 1, wherein the at least one pharmaceutical auxiliary is selected from the group consisting of acidifying agents, intense sweeteners, aromas, flavor enhancers, dyes, thickeners, surfactants and finely divided pigments.

13. The mixture according to claim 1, wherein the mixture is in the form of agglomerates.

14. The mixture according to claim 13, wherein the agglomerates have a particle size of from 0.063 to 500 μm.

15. The mixture according to claim 1, comprising:
a) 70-95% by weight of said agglomerated isomalt ST or said isomalt GS,
b) 2-15% by weight of said crosslinked polyvinylpyrrolidone,
c) 0.1-15% by weight of said lubricant,
d) 1-10% by weight of said water-insoluble, film-forming polymers,
e) 0-2% by weight of said water-soluble polyvinylpyrrolidone, and
f) 0-15% by weight of at least one said pharmaceutical auxiliaries auxiliary,
wherein the sum of components a) to f) is 100% by weight.

16. The mixture according to claim 1, comprising:
a) 85-95% by weight of said isomalt ST or said isomalt GS,
b) 3-10% by weight of said crosslinked polyvinylpyrrolidone,
c) 0.1-5% by weight of said lubricant,
d) 1-10% by weight of said polyvinylacetate,
e) 0-2% by weight of said water-soluble polyvinylpyrrolidone, and
f) 0-15% by weight of at least one said pharmaceutical auxiliary,
wherein the sum of components a) to f) is 100% by weight.

17. The mixture according to claim 1, wherein the mixture is in the form of a pharmaceutical formulation.

18. A tablet comprising a mixture according to claim 1, wherein the tablet has a disintegration time in an aqueous medium of less than or equal to 25 seconds.

19. The tablet according to claim 18, wherein the tablet has a breaking strength of greater than or equal to 50 N.

20. The tablet according to claim 18, comprising 20 to 99% by weight, based on the total tablet weight, of the mixture.

21. The tablet according to claim 18, comprising more than one pharmaceutical auxiliary.

22. The tablet according to claim 18, wherein the tablet is coated with isomalt GS.

23. A process for the preparation of a mixture according to claim 1, wherein agglomerated isomalt and crosslinked polyvinylpyrrolidone are agglomerated with an aqueous dispersion of the water-insoluble polymer in the presence of the lubricant.

24. The process according to claim 23, wherein agglomerated isomalt is agglomerated with an aqueous dispersion of the water-insoluble polymer, which additionally comprises crosslinked polyvinylpyrrolidone in suspended form, in the presence of the lubricant.

25. The process according to claim 23, wherein the agglomeration takes place in a fluidized-bed granulator, a mixer or a spray tower.

26. The mixture according to claim 4, wherein isomalt GS comprises 6-O-α-D-glucopyranosyl-D-sorbitol (1,6-GPS) in an amount of 75% by weight, and 1-O-α-D-glucopyranosyl-D-mannitol (1,1-GPM) in an amount of 25% by weight (in each case based on dry substance of the isomalt GS).

27. The mixture according to claim 5, wherein a maximum particle size of the isomalt is selected from the group consisting of at most 50 μm, at most 40 μm, at most 35 μm and at most 30 μm.

28. The mixture according to claim 8, wherein the lubricant comprises magnesium stearate together with polyethylene glycol.

29. The mixture according to claim 14, wherein the agglomerates have a particle size of from 63 to 500 μm.

30. The tablet according to claim 19, comprising 20 to 99% by weight, based on the total tablet weight, of the mixture.
